(19) United States
(12) Patent Application Publication
(10) Pub. No.: US 2007/0196513 A1
(43) Pub. Date: Aug. 23, 2007

(54) METHODS AND COMPOSITIONS FOR IMPROVING CARDIOVASCULAR RISK FACTORS AND METABOLIC RISK FACTORS THAT CAUSE SYNDROME X

(76) Inventor: Shil C. Kothari, Fairfield, CA (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(21) Appl. No.: 11/534,629

(22) Filed: Sep. 22, 2006

Related U.S. Application Data
(63) Continuation of application No. 11/360,559, filed on Feb. 22, 2006.

(51) Int. Cl. A61K 36/185; A61K 31/714; A61K 31/555; A61K 31/525; A61K 36/82; A61K 33/04; A61K 38/16

(52) U.S. Cl. 424/702; 424/725; 424/729; 514/251; 514/52; 514/350; 514/184; 514/12

(57) ABSTRACT

Compositions and related methods of Cissus quadrangularis plant extracts and soy albumin that improve various cardiovascular risk factors and metabolic risk factors that cause Syndrome X. In one exemplary embodiment, university studies demonstrated that a formulation of 300 mg Cissus quadrangularis plant extract, 3 mg niacin-bound chromium, 500 mg of green tea extract, 24 mg of selenium, 100 mg of soy albumin, 100 mg of vitamin B6, 0.1 mg of vitamin B12 and 0.8 mg of folic acid to obese and overweight humans daily for eight weeks provided unexpected synergistic results that improved a variety of health related factors. In particular, supplementation with the Cissus quadrangularis formula resulted in significant weight and fat loss, decreased systolic and diastolic blood pressure, decreased total and LDL cholesterol levels and triglycerides, C-reactive protein and fasting blood glucose levels, improved total cholesterol/HDL and LDL/HDL ratios, increased HDL cholesterol, serum serotonin, creatinine and urinary malondialdehyde levels, and improved fat burning mechanisms.

### RELATED APPLICATION

This is a continuation application of U.S. patent application Ser. No. 11/360,559 filed Feb. 22, 2006, the contents of which are incorporated by reference herein in its entirety.

### BACKGROUND OF THE INVENTION

The present disclosure relates generally to compositions and related methods improve a variety of health related factors. More specifically, the present disclosure relates to use of extracts of one or more Cissus quadrangularis plants and soy albumin to improve various cardiovascular and metabolic risk factors that are known to cause Syndrome X and provide a variety of related health benefits.

### DESCRIPTION OF THE RELATED ART

Syndrome X (or metabolic syndrome) is a well-known syndrome, which is often defined by obesity, cardiovascular disease and insulin resistance or diabetes. The American Heart Association (AHA) has indicated that Syndrome X in humans is characterized by a group of metabolic risk factors. More specifically, the AHA identified the following metabolic risk factors as contributing to Syndrome X:
1. Abdominal obesity (excessive fat tissue in and around the abdomen);
2. Atherogenic dyslipidemia (blood fat disorders—high triglycerides, low HDL cholesterol and high LDL cholesterol—that foster plaque buildups in artery walls);
3. Elevated blood pressure;
4. Insulin resistance or glucose intolerance (the body can't properly use insulin or blood sugar);
5. Prothrombotic state (e.g., high fibrinogen or plasminogen activator inhibitor—1 in the blood); and
6. Proinflammatory state (e.g., elevated C-reactive protein in the blood).

(See http://www.americanheart.org/presenter.jhtml?identifier=4756). The AHA and others have also acknowledged that people with Syndrome X have an increased risk of coronary heart disease and other diseases related to plaque buildups in artery walls (e.g., stroke and peripheral vascular disease) and type 2 diabetes. Unfortunately, Syndrome X has become increasingly common in the United States. The AHA has estimated that about 20-25 percent of the adults in the United States (over 50 million Americans) have Syndrome X.

The AHA also has indicated that the dominant underlying risk factors for Syndrome X appear to be abdominal obesity and insulin resistance. Insulin resistance is a generalized metabolic disorder, in which the body can't use insulin efficiently. This is why Syndrome X is also called insulin resistance syndrome. Other metabolic risk factors for Syndrome X include physical inactivity, aging and hormonal imbalance.

Unfortunately, some people are genetically predisposed to insulin resistance. It is well accepted that acquired metabolic risk factors, such as excess body fat and physical inactivity, can elicit insulin resistance and result in Syndrome X in these people. For individuals that are genetically predisposed to insulin resistance and, as a result, predisposed to acquiring Syndrome X, it is especially important to improve, control or eliminate the metabolic risk factors thought to cause insulin resistance and Syndrome X. It is believed that most people with insulin resistance have abdominal obesity. Accordingly, it is believed that abdominal obesity may play a larger role in causing insulin resistance and Syndrome X than other metabolic risk factors. It is well known that abdominal obesity can be controlled or reduced by overall weight loss and reduction of BMI.

Unfortunately, at the present, the biologic mechanisms of insulin resistance at the molecular level and how certain metabolic risk factors act to cause insulin resistance and Syndrome X aren't fully understood and appear to be complex. Similarly, at the present, the biological mechanisms of Syndrome X are also complex and not fully understood.

Presently, there is no universally-accepted criteria for diagnosing the Syndrome X. The AHA acknowledges that the criteria proposed by the National Cholesterol Education Program (NCEP) Adult Treatment Panel III (ATP III), are the most current and widely used criteria for diagnosing Syndrome X.

Additionally, the AHA and the National Heart, Lung, and Blood Institute recommend that the Syndrome X or metabolic syndrome be identified or diagnosed by the presence of three or more of the following metabolic risk factors:
1. Central obesity as measured by an elevated waist circumference:
   Men—Equal to or greater than 40 inches (102 cm)
   Women—Equal to or greater than 35 inches (88 cm)
2. Elevated triglycerides:
   Equal to or greater than 150 mg/dL
   Reduced HDL (“good”) cholesterol:
      Men—Less than 40 mg/dL
      Women—Less than 50 mg/dL
3. elevated Blood pressure:
   Equal to or greater than 130/85 mm/Hg
4. Elevated fasting glucose:
   Equal to or greater than 100 mg/dL

(See http://www.americanheart.org/presenter.jhtml?identifier=534). Accordingly, control and improvement of these and related metabolic risk factors prevent the onset and treat Syndrome X. The AHA has indicated that to gain the most benefit from modifying multiple metabolic risk factors that cause Syndrome X, the underlying insulin resistant state must become a target of therapy and that the safest, most effective and preferred way to reduce insulin resistance in overweight and obese people is through weight loss and increased physical activity. The AHA has also indicated that the following steps are also important for managing Syndrome X:
1. Routinely monitoring body weight (especially the index for central obesity), blood glucose, lipoproteins and blood pressure.
2. Treating individual risk factors (hyperlipidemia, hypertension and high blood glucose) according to established guidelines.
3. Carefully choosing anti-hypertensive drugs because different agents have different effects on insulin sensitivity.

(See http://www.americanheart.org/presenter.jhtml?identifier=534). Accordingly, improving and thereby eliminating these and other related metabolic risk factors that cause Syndrome X, is an effective way of preventing, treating, and controlling the progression of Syndrome X.

A related condition that effects most Americans is cardiovascular disease. In fact, cardiovascular disorders are the number one killer of both men and women in the United States. Coronary heart disease is so prevalent that it is estimated that one in five Americans have some form of it. It is also estimated that as many as 1.1 million Americans will have a coronary attack this year and about one-third of them will die from that attack.

The AHA has acknowledged that common cardiovascular risk factors for coronary heart disease and stroke that can be controlled or treated include high total cholesterol (240 mg/dL or higher), high triglyceride levels (blood fats, 150 mg/dL or higher), high LDL cholesterol levels (greater than 100 mg/dL), low HDL cholesterol levels (less than 40 mg/dL for men; less than 50 mg/dL for women), high blood pressure (135/85 mm/Hg or higher), smoking, diabetes, physical inactivity, and being overweight (BMI of 25.1 to 30.0) or obese (BMI of 30.0 or greater). (See http://strokeassociation.org/presenter.jhtml?identifier=3027394 & http://strokeassociation.org/presenter.jhtml?identifier=4716 which are incorporated herein by reference). It is well accepted that these cardiovascular risk factors cause a variety of ailments, including cardiovascular disease and death. Accordingly, in an effort to prevent or treat these ailments and prolong life, improvement and elimination of these and related cardiovascular risk factors is highly desirable.

Various treatments have been created to target improvement or elimination of the treatable cardiovascular risk factors that cause various coronary ailments and the metabolic risk factors that cause Syndrome X. Unfortunately, none of these treatments have been shown to be particularly effective at improving or eliminating either the cardiovascular risk factors (elevated total cholesterol, triglyceride, LDL cholesterol, blood pressure, reduced HDL cholesterol, diabetes and being overweight of obese) or the metabolic risk factors (obesity, elevated triglycerides, fasting glucose levels and blood pressure, and reduced HDL) that cause Syndrome X to such a degree that they are significantly improved or eliminated and, in the case of the metabolic risk factors, Syndrome X is prevented or treated. Additionally, the known treatments can often be difficult to administer, cause serious and undesirable side effects and often become less and less effective over time.

Additionally, it is a well known fact that the incidence of obesity in adults as well as children, a known risk factor that causes Syndrome X, is increasing globally. Once considered a problem of developed countries, this global epidemic also affects developing countries. Coupled with this epidemic are obesity-related complications such as cardiovascular disease, stroke, depression, and Type-2 diabetes, all of which are spreading rapidly across poor and middle-income countries, where infectious diseases and malnutrition have previously overshadowed such illnesses. In Cameroon, Africa about 25% of the adult population is either overweight or obese. This figure, though alarming, has followed a similar trend to Europe and the United States, where there has been a consistent increase in the past 20 years. In 1991, four states in the United States have obesity prevalence rates of 15-19% and none had rates above 20%. In 2004, seven states had obesity prevalence rates of 15-19%, 33 states had rates of 20-24%, and nine states had rates more than 25%.

Associations between obesity and high blood pressure, high blood cholesterol, and low levels of high density lipoprotein-cholesterol (HDL-C) have been shown in men and women in diverse race/ethnic groups. Associations have also been reported between obesity and high fasting blood glucose levels. This perturbation of blood lipid components as well as blood glucose levels is referred to by some as Syndrome X.

A number of anorexiant pharmacological agents, such as sibutramine, enhance satiation by blocking the reuptake of monoamine neurotransmitters (serotonin, norepinephrine, and to a lesser extent, dopamine) in the hypothalamus. Blocking monoamine reuptake increases the signal transmitted to the postsynaptic nerve resulting in satiation or satiety. Another common pharmacological mode used to treat obesity employs drugs designed to block the absorption of dietary fat, such as orlistat. Orlistat binds to gastric, pancreatic, and carboxyl-ester lipases in the gut lumen and blocks the digestion of dietary fat by preventing lipase from interacting with its lipid target. Among other drawbacks, the inhibition of fat digestion decreases mixed micelle formation and absorption of long-chain fatty acids, cholesterol, and certain fat-insoluble vitamins.

The above pharmacologic therapies in combination with dietary regimes have been used in obese patients to produce weight loss and attempt to reverse the accompanying complications and conditions that are associated with them. Unfortunately, these methods are not only compromised by potential safety issues, which has sometimes led to their removal from the market, they often are not very effective, lose their effectiveness over time, can cause numerous undesirable side effects and complications and are expensive.

Accordingly, compositions and related methods that improve or eliminate well known cardiovascular risk factors and the metabolic risk factors that cause Syndrome X are needed. Additionally, compositions and related methods that provide additional health benefits, including but not limited to, reducing weight, body fat, blood pressure, cholesterol, triglyceride, and fasting blood glucose levels, increasing or improving fat burning mechanisms, reducing C-reactive protein levels, providing appetite suppression and preventing binge eating and emotional eating and increasing serum serotonin, urinary malondialdehyde and creatinine levels are also needed. The present disclosure provides these and other related advantages.

### SUMMARY OF THE INVENTION

Briefly, and in general terms, by way of example and not limitation, one embodiment of the present disclosure resides in composition and related methods using extracts from the Cissus quadrangularis plant and soy albumin to improve or eliminate cardiovascular risk factors and the metabolic risk factors that cause Syndrome X, thereby preventing or treating Syndrome X. Additionally, by way of example and not limitation, one aspect of the present disclosure resides in using Cissus quadrangularis plant extracts and soy albumin to improve or eliminate at least one cardiovascular risk factor or metabolic risk factor that causes Syndrome X.

Another aspect of the present disclosure resides in improved compositions and related methods using extracts from the Cissus quadrangularis plant, soy albumin, selenium and chromium to improve or eliminate cardiovascular risk factors and the metabolic risk factors that cause Syndrome X, thereby preventing or treating Syndrome X. Additionally, by way of example and not limitation, yet another aspect of the present disclosure resides in using Cissus quadrangularis plant extracts, soy albumin, selenium, and chromium to improve or eliminate at least one cardiovascular risk factor or metabolic risk factor that causes Syndrome X.

In yet another aspect of the present disclosure resides in improved compositions and related methods using extracts from the Cissus quadrangularis plant, soy albumin, selenium, chromium, green tea extract, vitamin B6, vitamin B12 and folic acid to improve or eliminate cardiovascular risk factors and the metabolic risk factors that cause Syndrome X, thereby preventing or treating Syndrome X. Additionally, by way of example and not limitation, yet another aspect of the present disclosure resides in using Cissus quadrangularis plant extracts, soy albumin, selenium, chromium, green tea extract, vitamin B6, vitamin B12, and folic acid to improve or eliminate at least one cardiovascular risk factor or metabolic risk factors that cause Syndrome X.

By way of example and not limitation, in another exemplary embodiment of the present disclosure, a method for improving or eliminating cardiovascular risk factors is provided. The method includes providing a composition containing an effective amount of extracts from one or more Cissus quadrangularis plants and soy albumin to a mammal to improve or eliminate one or more cardiovascular risk factors and claiming that the composition improves or eliminates one or more cardiovascular risk factors in a mammal.

By way of example and not limitation, in yet another exemplary embodiment of the present disclosure, a method for improving or eliminating cardiovascular risk factors is provided. The method includes providing a composition containing an effective amount of extracts from one or more Cissus quadrangularis plants, soy albumin, selenium, and chromium to a mammal to improve or eliminate one or more cardiovascular risk factors and claiming that the composition improves or eliminates one or more cardiovascular risk factors in a mammal.

By way of example and not limitation, in yet another exemplary embodiment of the present disclosure, a method for improving or eliminating cardiovascular risk factors is provided. The method includes providing a composition containing an effective amount of extracts from one or more Cissus quadrangularis plants, soy albumin, selenium, chromium, and at least two components selected from the group of green tea extract, vitamin B6, vitamin B12 and folic acid, to a mammal to improve or eliminate one or more cardiovascular risk factors and claiming that the composition improves or eliminates one or more cardiovascular risk factors in a mammal.

By way of example and not limitation, in yet another exemplary embodiment of the present disclosure, a method for improving or eliminating cardiovascular risk factors is provided. The method includes providing a composition containing an effective amount of extracts of one or more Cissus quadrangularis plants and soy albumin to a mammal to improve or eliminate one or more metabolic risk factors that cause Syndrome X and claiming that the composition improves or eliminates Syndrome X or one or more of the metabolic risk factors that cause Syndrome X in a mammal.

By way of example and not limitation, yet another aspect of the present disclosure relates to providing a mammal with an effective amount of Cissus quadrangularis extracts and soy albumin to provide the mammal with one or more of the following benefits: (1) increased weight loss and reduced BMI, (2) increased or improved fat burning mechanisms, (3) appetite suppression and prevent binge eating and/or emotional eating, (4) increased serum serotonin levels, (5) increased urinary malondialdehyde levels, (6) increased creatinine levels, (7) reduced total cholesterol, LDL cholesterol, triglyceride and systolic, diastolic and total blood pressure levels, (8) increased HDL or “good” cholesterol levels, (9) reduced fasting blood sugar levels, (10) prevention or treatment of Syndrome X, and (11) reducing C-reactive protein.

By way of example and not limitation, yet another aspect of the present disclosure relates to providing a mammal with an effective amount of Cissus quadrangularis extracts, soy albumin, selenium and chromium to provide the mammal with one or more of the following benefits (1) increased weight loss and reduced BMI, (2) increased or improved fat burning mechanisms, (3) appetite suppression and prevent binge eating and/or emotional eating, (4) increased serum serotonin levels, (5) increased urinary malondialdehyde levels, (6) increased creatinine levels, (7) reduced total cholesterol, LDL cholesterol, triglyceride and systolic, diastolic and total blood pressure levels, (8) increased HDL or “good” cholesterol levels, (9) reduced fasting blood sugar levels, (10) prevention or treatment of Syndrome X, and (11) reducing C-reactive protein.

By way of example and not limitation, yet another aspect of the present disclosure relates to providing a mammal with an effective amount of Cissus quadrangularis extracts, soy albumin, selenium, chromium, green tea extract, vitamin B6, vitamin B12 and folic acid to provide the mammal with one or more of the following benefits (1) increased weight loss and reduced BMI, (2) increased or improved fat burning mechanisms, (3) appetite suppression and prevention of binge eating and/or emotion eating, (4) increased serum serotonin levels, (5) increased urinary malondialdehyde levels, (6) increased creatinine levels, (7) reduced total cholesterol, LDL cholesterol, triglyceride and systolic, diastolic and total blood pressure levels, (8) increased HDL or “good” cholesterol levels, (9) reduced fasting blood sugar levels, (10) prevention or treatment of Syndrome X, and (11) reducing C-reactive protein.

By way of example and not limitation, yet another aspect of the present disclosure relates to using a composition containing an effective amount of extracts from one or more Cirrus quadrangularis plants and soy albumin to improve one or more cardiovascular risk factors in a mammal.

By way of example and not limitation, yet another aspect of the present disclosure relates to using a composition containing an effective amount of extracts from one or more Cissus quadrangularis plants, soy albumin, selenium and chromium to improve one or more cardiovascular risk factors in a mammal.

By way of example and not limitation, yet another aspect of the present disclosure relates to using a composition containing an effective amount of extracts from one or more Cissus quadrangularis plants, soy albumin, selenium, chromium, green tea extract, vitamin B6, vitamin B12, and folic acid to improve one or more cardiovascular risk factors in a mammal.

By way of example and not limitation, in yet another detailed aspect of the present disclosure, the effective amount of the Cissus quadrangularis plant extracts if 100 mg to 900 mg and the effective amount of soy albumin is 10 mg to 400 mg.

By way of example and not limitation, in yet another detailed aspect of the present disclosure, the effective amount of the Cissus quadrangularis plant extracts provides 2.5 mg to 15 mg of active Cissus quadrangularis and the effective amount of soy albumin provides 50 mg to 200 mg of active soy albumin.

By way of example and not limitation, in yet another detailed aspect of the present disclosure, the effective amount of chromium is 1 mg to 6 mg and the effective amount of selenium is 5 mg to 100 mg.

By way of example and not limitation, in yet another detailed aspect of the present disclosure, the composition includes an effective amount of Cissus quadrangularis plant extracts, soy albumin and an effective amount of at least three items from the group of green tea extract, vitamin B6, vitamin B12, and folic acid.

By way of example and not limitation, in yet another detailed aspect of the present disclosure, the effective amount of Cissus quadrangularis plant extracts if 100 mg to 900 mg daily, the effective amount of soy albumin is 10 mg to 400 mg daily, the effective amount of chromium is 1 mg to 6 mg daily, the effective amount of selenium is 5 mg to 100 mg daily, the effective amount of green tea extract is 100 mg to 1000 mg daily, the effective amount of vitamin B6 is 10 mg to 400 mg daily, the effective amount of vitamin B12 is 0.01 mg to 1.0 mg daily and the effective amount of folic acid is 0.2 mg to 4.0 mg daily.

By way of example and not limitation, in yet another detailed aspect of the present disclosure, the effective amount in the composition provides 300 mg of Cissus quadrangularis plant extracts, 100 mg soy albumin, 3 mg niacin-bound chromium, 24 mg selenium, 500 mg green tea extract, 100 mg vitamin B6, 0.1 mg vitamin B12 and 0.8 mg folic acid daily.

By way of example and not limitation, in yet another detailed aspect of the present disclosure, the composition improves more than one cardiovascular risk factor in a mammal. Cardiovascular risk factors are well known in the art and include, but are not limited to: (1) weight, (2) BMI, (3) total cholesterol, (4) LDL cholesterol, (5) HDL cholesterol, (6) total cholesterol/HDL ratio, (7) LDL/HDL ratio, (8) serum serotonin levels, (9) systolic and diastolic blood pressure, (10) creatinine levels, (11) urinary malondialdehyde levels, (12) C-reactive protein levels, (13) triglyceride levels, and (14) fasting blood glucose levels.

By way of example and not limitation, in yet another detailed aspect of the present disclosure, the composition improves cardiovascular risk factors by providing one or more of the following benefits (1) decreasing weight and BMI, (2) lowering total cholesterol levels, (3) lowering systolic blood pressure, (4) lowering diastolic blood pressure, (5) decreasing LDL cholesterol levels, (6) increasing HDL cholesterol levels, (7) decreasing overall blood pressure, (8) lowering triglyceride levels, (9) increasing serum serotonin levels, (10) lowering fasting blood glucose levels, (11) increasing creatinine levels, (12) lowering C-reactive protein levels and (13) increasing urinary malondialdehyde levels.

By way of example and not limitation, in yet another detailed aspect of the present disclosure, by improving one or more of the cardiovascular risk factors, the composition treats Syndrome X or prevents the onset of Syndrome X.

By way of example and not limitation, in yet another detailed aspect of the present disclosure, the composition treats Syndrome X or prevents the onset of Syndrome X by eliminating one or more of the metabolic risk factor that cause Syndrome X.

By way of example and not limitation, in yet another detailed aspect of the present disclosure, the chromium is niacin-bound chromium.

### BRIEF DESCRIPTION OF THE DRAWINGS

FIG. 1 shows a bar graph which summarizes the results of an experiment demonstrating that eight weeks of supplementation with the Cissus quadrangularis formula caused a significant and unexpected decrease in percentage of fat in a group of obese human subjects not on calorie restricted diets and a group of obese human subjects on calorie restricted diets, when compared to their respective control groups. The results also showed significant and unexpected decrease in the percentage of fat in a group of overweight human subjects that were not on calorie restricted diets.

FIG. 2 shows a bar graph which summarizes the results of an experiment demonstrating that eight weeks of supplementation with the Cissus quadrangularis formula caused a significant and unexpected weight loss in a group of obese human subjects not on calorie restricted diets and a group of obese human subjects on calorie restricted diets, when compared to their respective control groups. The results also shows significant and unexpected weight loss in a group of overweight human subjects that were not on calorie restricted diets.

FIG. 3 shows a bar graph which summarizes the results of an experiment demonstrating that eight weeks of supplementation with the Cissus quadrangularis formula caused a significant and unexpected decrease in the systolic blood pressure levels in a group of obese human subjects not on calorie restricted diets and a group of obese human subjects on calorie restricted diets, when compared to their respective control groups. The results also showed a significant and unexpected decrease in the systolic blood pressure levels in a group of overweight human subjects that were not on calorie restricted diets.

FIG. 4 shows a bar graph which summarizes the results of an experiment demonstrating that eight weeks of supplementation with the Cissus quadrangularis formula caused a significant and unexpected decrease in the diastolic blood pressure levels in a group of obese human subjects not on calorie restricted diets and a group of obese human subjects on calorie restricted diets, when compared to their respective control groups. The results also showed a significant and unexpected decrease in the diastolic blood pressure levels in a group of overweight human subjects that were not on calorie restricted diets.

FIG. 5 shows a bar graph which summarizes the results of an experiment demonstrating that eight weeks of supplementation with the Cissus quadrangularis formula caused a significant and unexpected decrease in the total cholesterol levels in a group of obese human subjects not on calorie restricted diets and a group of obese human subjects on calorie restricted diets, when compared to their respective control groups. The results also shows a significant and unexpected decrease in the total cholesterol levels in a group of overweight human subjects that were not on calorie restricted diets.

FIG. 6 shows a bar graph which summaries the results of an experiment demonstrating that eight weeks of supplementation with the Cissus quadrangularis formula caused a significant and unexpected decrease in LDL cholesterol levels in a group of obese human subjects not on calorie restricted diets and a group of obese human subjects on calorie restricted diets, when compared to their respective control groups. The results also shows a significant and unexpected decrease in LDL cholesterol levels in a group of overweight human subjects that were not on calorie restricted diets.

FIG. 7 shows a bar graph which summarizes the results of an experiment demonstrating that eight weeks of supplementation with the Cissus quadrangularis formula caused a significant and unexpected increase in HDL cholesterol levels in a group of obese human subjects not on calorie restricted diets and a group of obese human subjects on calorie restricted diets, when compared to their respective control groups. The results also shows a significant and unexpected increase in HDL cholesterol levels in a group of overweight human subjects that were not on calorie restricted diets.

FIG. 8 shows a bar graph which summarizes the results of an experiment demonstrating that eight weeks of supplementation with the Cissus quadrangularis formula caused a significant and unexpected decrease in triglyceride levels in a group of obese human subjects not on calorie restricted diets and a group of obese human subjects on calorie restricted diets, when compared to their respective control groups. The results also shows a significant and unexpected decrease in triglyceride levels of a group in overweight human subjects that were not on calorie restricted diets.

FIG. 9 shows a bar graph which summarizes the results of an experiment demonstrating that eight weeks of supplementation with the Cissus quadrangularis formula caused a significant and unexpected decrease in the total cholesterol/HDL ratio in a group of obese human subjects not on calorie restricted diets and a group of obese human subjects on calorie restricted diets, when compared to their respective control groups. The results also showed a significant and unexpected decrease in the total cholesterol/HDL ratio in a group of overweight human subjects that were not calorie restricted diets.

FIG. 10 shows a bar graph which summarizes the results of an experiment demonstrating that eight weeks of supplementation with the Cissus quadrangularis formula caused a significant and unexpected decrease in the LDL/HDL ratio in a group of obese human subjects not on calorie restricted diets and a group of obese human subjects on calorie restricted diets, when compared to their respective control groups. The results also shows a significant and unexpected decrease in the LDL/HDL ratio in a group of overweight human subjects that were not calorie restricted diets.

FIG. 11 shows a bar graph which summarizes the results of an experiment demonstrating that eight weeks of supplementation with the Cissus quadrangularis formula caused a significant and unexpected decrease in the fasting blood sugar levels in a group of obese human subjects not on calorie restricted diets and a group of obese human subjects on calorie restricted diets, when compared to their respective control groups. The results also shows a significant and unexpected decrease in the fasting blood sugar levels in a group of overweight human subjects that were not calorie restricted diets.

FIG. 12 shows a bar graph which summarizes the results of an experiment demonstrating that eight weeks of supplementation with the Cissus quadrangularis formula caused a significant and unexpected increase in the serum serotonin levels in a group of obese human subjects not on calorie restricted diets and a group of obese human subjects on calorie restricted diets, when compared to their respective control groups. The results also showed a significant and unexpected increase in the serum serotonin levels in a group of overweight human subjects that were not calorie restricted diets.

FIG. 13 shows a bar graph which summarizes the results of an experiment demonstrating that eight weeks of supplementation with the Cissus quadrangularis formula caused a significant and unexpected increase in creatinine levels in a group of obese human subjects not on calorie restricted diets and a group of obese human subjects on calorie restricted diets, when compared to their respective control groups. The results also showed a significant and unexpected increase in creatinine levels in a group of overweight human subjects that were not calorie restricted diets.

FIG. 14 shows a bar graph which summarizes the results of an experiment demonstrating that eight weeks of supplementation with the Cissus quadrangularis formula caused a significant and unexpected decrease in C-reactive protein levels in a group of obese human subjects not on calorie restricted diets and a group of obese human subjects on calorie restricted diets, when compared to their respective control groups. The results also shows a significant and unexpected decrease in C-reactive protein levels in a group of overweight human subjects that were not calorie restricted diets.

FIG. 15 shows a bar graph which summarizes the results of an experiment demonstrating that eight weeks of supplementation with the Cissus quadrangularis formula caused a significant and unexpected decrease in urinary malondialdehyde levels in a group of obese human subjects not on calorie restricted diets and a group of obese human subjects on calorie restricted diets, when compared to their respective control groups. The results also showed a significant and unexpected decrease in urinary malondialdehyde levels in a group of overweight human subjects that were not calorie restricted diets.

FIG. 16 shows a summary table which summarizes the results of an experiment demonstrating that eight weeks of supplementation with the Cissus quadrangularis formula caused significant and unexpected beneficial effects on weight, systolic and diastolic blood pressure, total cholesterol, LDL and HDL cholesterol, and cholesterol in a group of obese human subjects not on calorie restricted diets and a group of obese human subjects on calorie restricted diets, when compared to their respective control groups. The results also shows similar significant and unexpected beneficial effects on the indicated parameters in a group of overweight human subjects that were not calorie restricted diets.

FIG. 17 shows a summary table which summarizes the results of an experiment demonstrating that eight weeks of supplementation with the Cissus quadrangularis formula caused significant and unexpected beneficial effects on total cholesterol/HDL ratio, LDL/HDL ratio, serum serotonin levels, fasting blood glucose levels, creatinine levels, urinary malondialdehyde levels, and C-reactive protein levels in a group of obese human subjects not on calorie restricted diets and a group of obese human subjects on calorie restricted diets, when compared to their respective control groups. The results also shows similar significant and unexpected beneficial effects on the indicated parameters in a group of overweight human subjects that were not calorie restricted diets.

### DETAILED DESCRIPTION OF THE PREFERRED EMBODIMENTS

Briefly, and in general terms, the present disclosure is directed to providing compositions and related methods for improving or eliminating a wide variety of cardiovascular and metabolic risk factors that are known to cause numerous ailments, including Syndrome X.

In one exemplary embodiment of the present disclosure, compositions and related methods containing effective amount of extracts from the Cissus quadrangularis plant and soy albumin are shown to improve or eliminate cardiovascular risk factors and the metabolic risk factors that cause Syndrome X, thereby preventing or treating Syndrome X. Additionally, the compositions and related methods of the present disclosure can be used to improve to eliminate at least one cardiovascular risk factor or metabolic risk factors that causes Syndrome X. The Cissus quadrangularis plant and soy albumin together act synergistically, in an unexpected way, to significantly improve or eliminate cardiovascular or metabolic risk factors that cause Syndrome X, thereby preventing or treating Syndrome X.

In another exemplary embodiment of the present disclosure, the composition contains an effective amount of Cissus quadrangularis plant extracts, soy albumin, selenium and chromium, which together act synergistically, in an unexpected way, to improve or eliminate at least one cardiovascular or metabolic risk factors that cause Syndrome X.

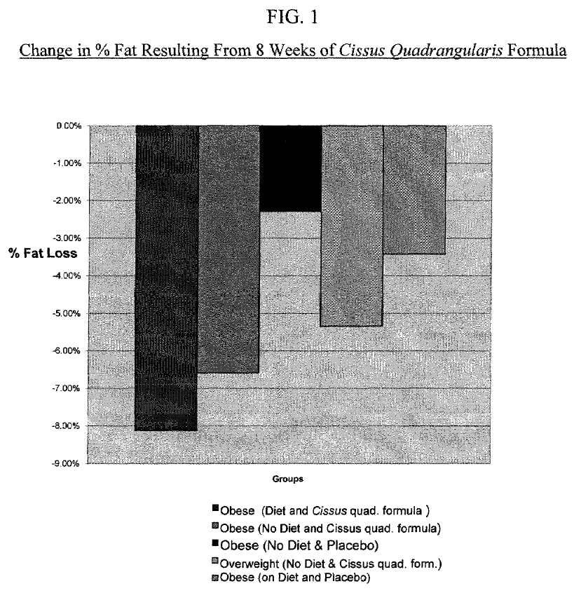

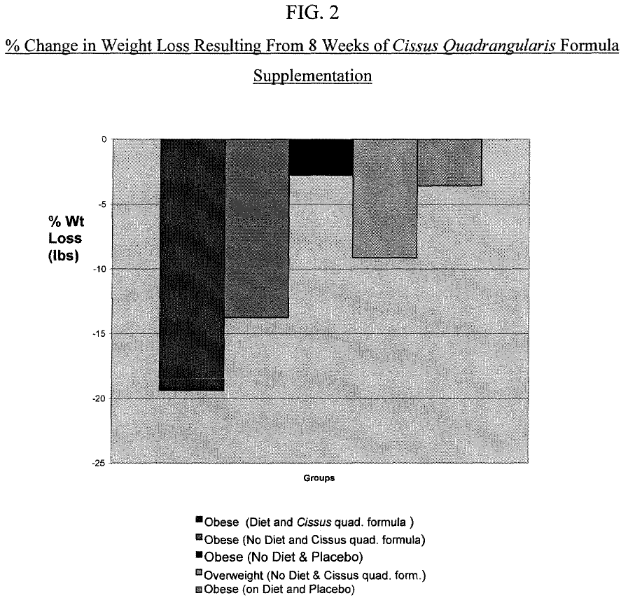

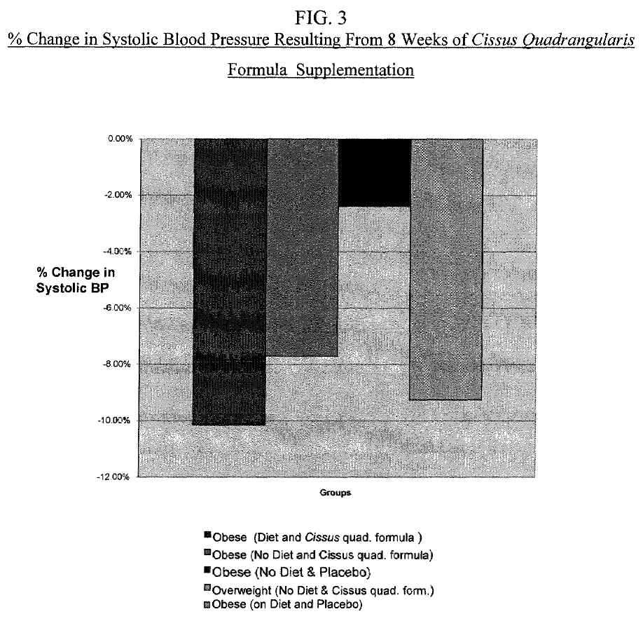

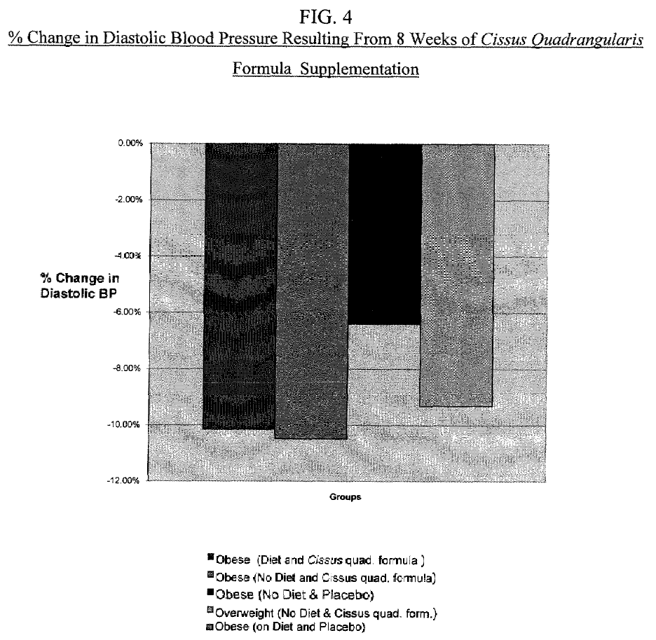

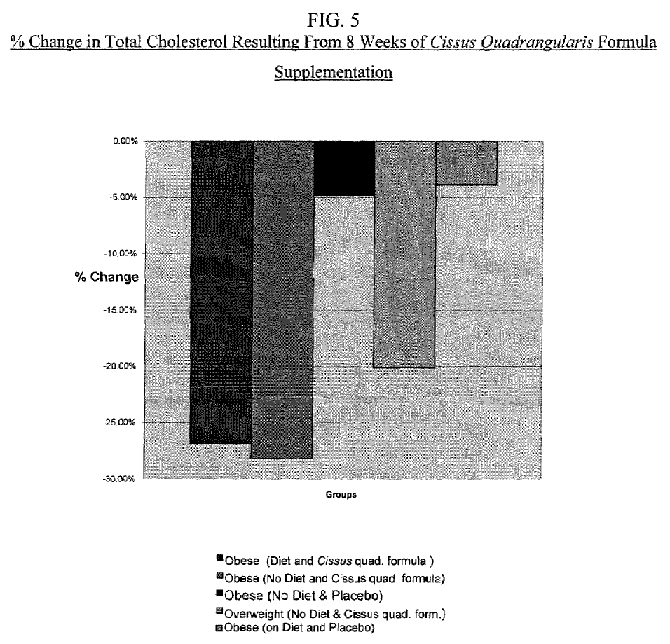

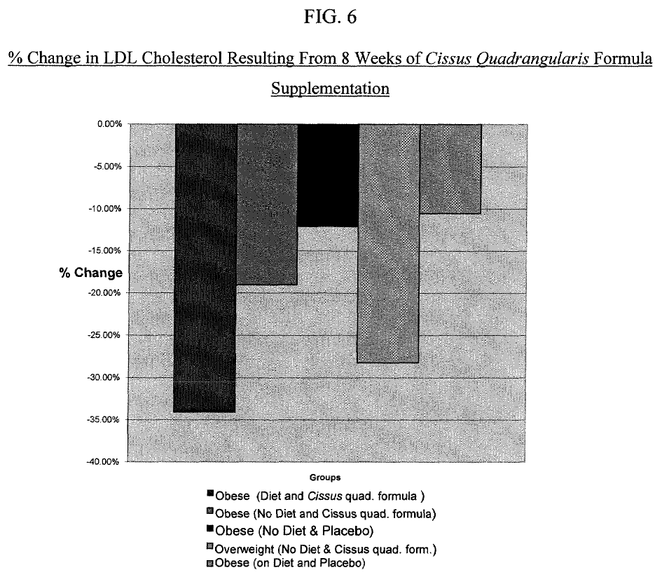

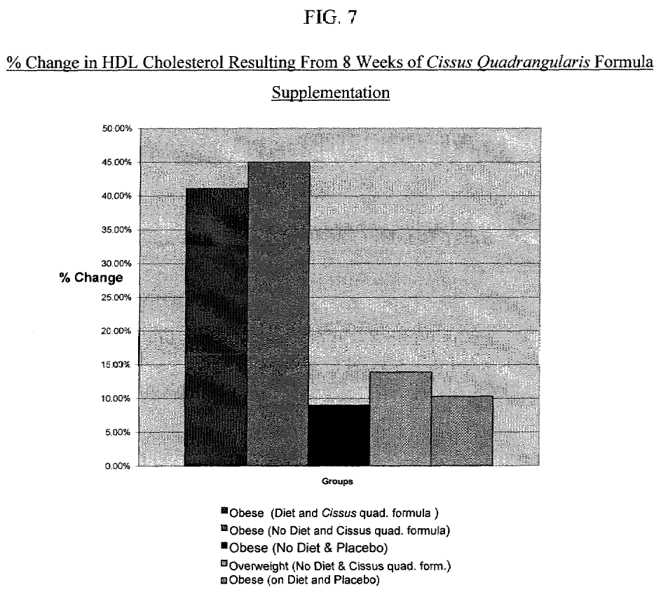

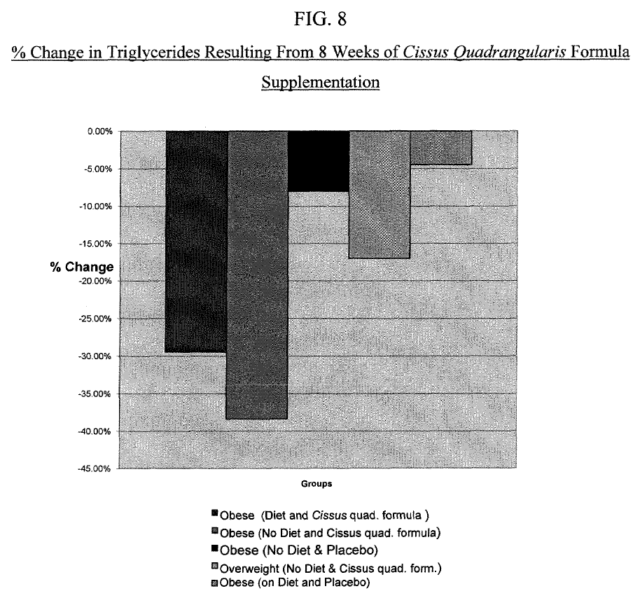

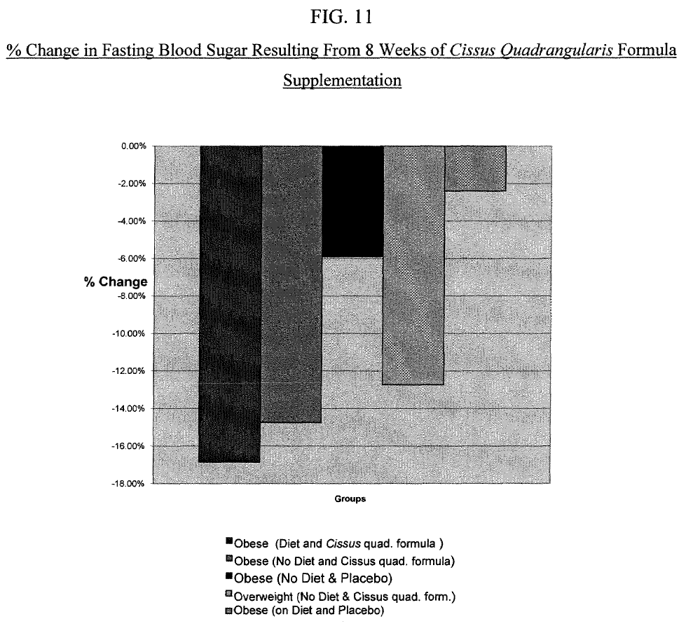

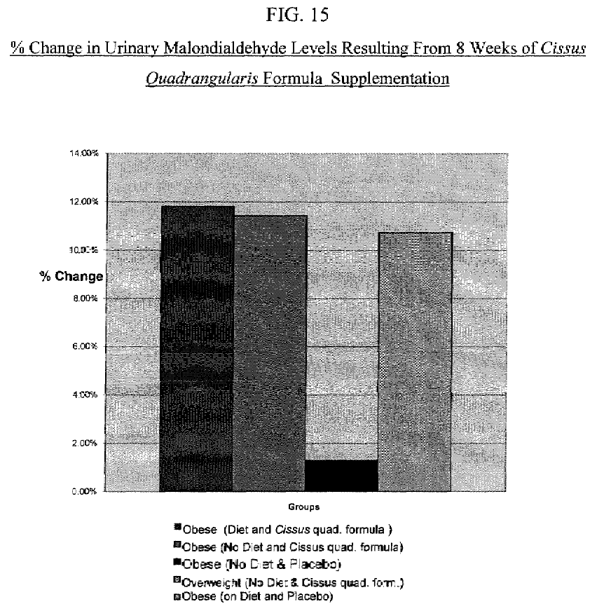

In yet another exemplary embodiment of the present disclosure, the composition contains an effective amount of extracts from the Cissus quadrangularis plant, soy albumin, selenium, chromium, green tea extract, vitamin B6, vitamin B12, and folic acid to improve or eliminate the cardiovascular and metabolic risk factors that cause Syndrome X. The components of the composition act synergistically, in an unexpected way, to improve or eliminate at least one of the cardiovascular or metabolic risk factors that cause Syndrome X. The results of the experiments shown in FIGS. 1-17 demonstrate that an effective amount of a composition containing these components provides significant unexpected improvements to a variety of health related factors and metabolic and cardiovascular risk factors that are known to cause Syndrome X.

Additionally, in another exemplary embodiment of the present disclosure, a method for improving or eliminating cardiovascular risk factors is provided. The method includes providing a composition containing an effective amount of extracts from one or more Cissus quadrangularis plants and soy albumin, or extracts from one or more Cissus quadrangularis plants, soy albumin, selenium, and chromium or extracts from one or more Cissus quadrangularis plants, soy albumin, selenium, chromium, green tea extract, vitamin B6, vitamin B12 and folic acid to a mammal to improve or eliminate one or more cardiovascular risk factors and claiming that the composition improves or eliminates one or more cardiovascular risk factors in a mammal.

As the experiments and data discussed in detail below prove, the compositions and methods of the present disclosure provide a mammal with one or more of the following benefits: (1) increased weight loss and reduced BMI, (2) increased or improved fat burning mechanisms, (3) appetite suppression and prevention of binge eating and/or emotional eating, (4) increased serum serotonin levels, (5) increased urinary malondialdehyde levels, (6) increased creatinine levels, (7) reduced total cholesterol, LDL cholesterol, triglyceride and systolic, diastolic and total blood pressure levels, (8) increased HDL or “good” cholesterol levels, (9) reduced fasting blood sugar levels, (10) prevention or treatment of Syndrome X, and (11) reducing C-reactive protein levels.

In one detailed aspect of the present disclosure, the effective amount of the Cissus quadrangularis plant extracts in the composition provides a mammal with approximately 100 mg to 900 mg daily and the effective amount of soy albumin in the composition provides a mammal with approximately 10 mg to 400 mg daily.

In yet another detailed aspect of the present disclosure, the effective amount of the Cissus quadrangularis plant extracts provides 2.5 mg to 15 mg of active Cissus quadrangularis to a mammal daily and the effective amount of soy albumin provides 50 mg to 200 mg of active soy albumin to the mammal daily.

In yet another detailed aspect of the present disclosure, the effective amount of chromium provides 1 mg to 6 mg of chromium to a mammal daily and the effective amount of selenium provides 5 mg to 100 mg of selenium to a mammal daily.

In yet another detailed aspect of the present disclosure, the effective amount of green tea extract provides 100 mg to 1000 mg of green tea extract to a mammal daily, the effective amount of vitamin B6 provides 10 mg to 400 mg of vitamin B6, the effective amount of vitamin B12 provides 0.01 mg to 1.0 mg vitamin B12 and the effective amount of folic acid provides 0.2 mg to 4.0 mg of folic acid to the mammal daily.

In yet another detailed aspect of the present disclosure, the composition provides approximately 300 mg of Cissus quadrangularis plant extracts, 100 mg soy albumin, 3 mg niacin-bound chromium, 24 mg selenium, 500 mg green tea extract, 100 mg vitamin B6, 0.1 mg vitamin B12 and 0.8 mg folic acid to the mammal daily.

It should be appreciated that for all of the compositions disclosed in the present disclosure, the exact amounts Cissus quadrangularis plant extracts, soy albumin, chromium, selenium, green tea extract, vitamin B6, vitamin B12 and folic acid, if any, will depend on the weight, type and condition of the mammal to whom the composition is being provided. Additionally, it should be appreciated that is it within the scope of the present invention to provide the components of the compositions separately so that they can be taken separately. It is also within the scope of this invention to divide the composition or its components into different portions to provide an effective amount of the composition daily.

It should also be appreciated that the compositions and related method disclosed improve numerous health related factors in a mammal, including but not limited to cardiovascular and metabolic risk factors known to cause Syndrome X. These health factors are well known in the art and include, but are not limited to (1) weight, (2) BMI, (3) total cholesterol, (4) LDL cholesterol, (5) HDL cholesterol, (6) total cholesterol/HDL ratio, (7) LDL/HDL ratio, (8) serum serotonin levels, (9) systolic and diastolic blood pressure, (10) creatinine levels, (11) urinary malondialdehyde levels, (12) C-reactive protein levels, (13) triglyceride levels, and (14) fasting blood glucose levels.

The compositions and related methods of the present disclosure improve numerous health related factors, including cardiovascular and metabolic risk factors, by (1) decreasing weight and BMI, (2) lowering total cholesterol levels, (3) lowering systolic blood pressure, (4) lowering diastolic blood pressure, (5) decreasing LDL cholesterol levels, (6) increasing HDL cholesterol levels, (7) decreasing overall blood pressure, (8) lowering triglyceride levels, (9) increasing serum serotonin levels, (10) lowering fasting blood glucose levels, (11) increasing creatinine levels, (12) lowering C-reactive protein levels, and (13) increasing urinary malondialdehyde levels.

In yet another detailed aspect of the present disclosure, the chromium in the composition and related methods is niacin-bound chromium.

Formula Composition

In order to test and validate the efficacy of the compositions disclosed in the present disclosure, a variety of university conducted and controlled experiments were performed to measure the effects of a composition containing Cissus quadrangularis plant extracts, soy albumin, chromium, selenium, green tea extract, vitamin B6, vitamin B12 and folic acid on numerous health related factors. The raw materials for the composition were obtained from Soy Labs and Gateway Health Alliances, both in California. The exact formulation of the composition used in those experiments and the amounts provided are shown in Table 1.

TABLE 1

| | | Active | | Active | | Gross | |
|---|---|---|---|---|---|---|---|
| | | Amount | | Amount | | Amount | |
| | | Per | | Per Day (2 | | Per Day (2 | |
| Item | Description | Capsule | Unit | capsules) | Unit | capsules) | Unit |
| Cissus | 5% ketosteroids | 7.5 | mg | 15 | mg | 300 | mg |
| quadrangularis | | | | | | | |
| ChromeMate* | Niacin-Bound Chromium | 0.15 | mg | 0.3 | mg | 3 | mg |
| | (10% Cr) -- Concentrate | | | | | | |
| Green Tea Extract | 40% Polyphenols, 22% | 100 | mg | 200 | mg | 500 | mg |
| (High Caffeine) | EGCG, 40% Caffeine | | | | | | |
| Selenium | 0.5% 1-Selenomethionine | 0.06 | mg | 0.12 | mg | 24 | mg |
| AlbumaSoy | Soy Albumin | 50 | mg | 100 | mg | 100 | mg |
| Vitamin B6 | pyridoxine hydrochloride | 50 | mg | 100 | mg | 100 | mg |
| Vitamin B12 | cyanocobalamin | 0.05 | mg | 0.1 | mg | 0.1 | mg |
| Folic Acid | Folic Acid | 0.4 | mg | 0.8 | mg | 0.8 | mg |
| Total Per Unit | | 104.08 | | 265.645 | mg | 1027.9 | mg |

The experiments were performed on a population comprising voluntary male and female participants with BMIs between 25 and 35. The purpose, nature, and potential risks of the study were explained to all participants, who gave written informed consent before participation. The study was conducted in accordance with the Helsinki Declaration (1983 version).

Participants

A total of one hundred and twenty overweight and obese participants of both sexes aged between 19 and 50 years were selected from a group responding to a radio and poster advertisements. The BMIs of participants ranged from 25.0 to 35.7, and their weight ranged from 65.0 to 120.5 kg. After physical examination, which included measurement of blood pressure, participants with unusually elevated fasting blood glucose levels, who were pregnant or lactating, as well as those on any form of weight reducing medication were excluded. Also excluded were participants involved in intense exercise programs, any who had medical conditions known to affect serum lipids or who had a history of drug or alcohol abuse. The participants were then divided into the four groups in Table 2, based on their BMIs. Group 3 was prescribed and used an energy reduced diet during the eight weeks of the experiment.

TABLE 2

| | | | No. of |
|---|---|---|---|
| Group No. | Participants | Treatment | Participants |
| 1 | Males/Females | Placebo | 30 |
| | BMI > 30 | 8 weeks (56 | |
| | | days), no dietary | |
| | | restriction | |
| 2 | Males/Females | Formula | 30 |
| | BMI > 30 | composition | |
| | | 8 weeks (56 | |
| | | days), no dietary | |
| | | restriction | |
| 3 | Males/Females | Formula | 30 |
| | BMI > 30 | composition | |
| | | 8 weeks (56 | |
| | | days), 2000–2300 Kcal/ | |
| | | day | |
| | | diet | |
| 4 | Males/Females | Formula | 30 |
| | BMI range of | composition | |
| | 25–28 | 8 weeks (56 | |
| | | days), no dietary | |
| | | restriction | |
| | | | Total Number of |
| | | | participants = 120 |

Baseline Characteristics of Participants

The baseline characteristics for the participants are shown in Table 3 below. The data is shown as a mean with standard deviation in parentheses. The anthropomorphic characteristics were primary outcome measures and the serological characteristics were secondary outcome measures.

TABLE 3

| | BMI | | | |
|---|---|---|---|---|
| | | | | 25–29 |
| | Greater than 30 (Obese) | | | (Overweight) |
| | Treatment | | | |
| | | Formulation/ | Formulation/ | Formulation/ |
| | Placebo | no diet | Diet | no diet |
| | (n = 30) | (n = 31) | (n = 31) | (n = 31) |
| Demographic | | | | |
| Characteristics (no.) | | | | |
| Sex | | | | |
| Male | 14 | 15 | 14 | 15 |
| Female | 16 | 16 | 17 | 16 |
| Age (years)* | 39.9 (9.6) | 41.0 (9.4) | 40.9 (9.8) | 39.7 (9.9) |
| Race or ethnic group | | | | |
| West African | 30 | 31 | 31 | 31 |
| Family History of Diabetes | 2 | 1 | 0 | 2 |
| History of Gestational | 2 | 2 | 3 | 2 |
| Diabetes | | | | |
| Anthropomorphic | | | | |
| Characteristics | | | | |
| Weight (kg) | 95.6 (14.3) | 95.8 (8.0) | 95.3 (9.1) | 76.3 (7.9) |
| Fat (%) | 41.8 (5.2) | 46.5 (5.8) | 48.1 (3.0) | 5.8 (5.2) |
| Body Mass Index | 38.4 (1.6) | 37.7 (1.5) | 37.5 (1.0) | 27.3 (0.7) |
| (kg/m²) | | | | |
| Waist Circumference (cm) | 103.4 (8.1) | 98.6 (4.6) | 97.8 (5.7) | 86.9 (3.2) |
| Serological | | | | |
| Characteristics | | | | |
| Total Cholesterol | 160.8 (18.0) | 159.1 (30.7) | 171.0 (24.4) | 152.6 (26.9) |
| (mg/dl) | | | | |
| LDL Cholesterol (mg/dl) | 103.5 (14.9) | 99.8 (11.7) | 116.6 (15.4) | 101.6 (17.0) |
| HDL Cholesterol (mg/dl) | 32.7 (7.5) | 36.6 (8.0) | 38.6 (7.1) | 44.4 (9.8) |
| Triglycerides (mg/dl) | 142.5 (31.8) | 156.0 (25.3) | 144.9 (30.0) | 117.4 (25.3) |
| C-Reactive protein | 8.0 (1.1) | 8.0 (1.0) | 8.1 (0.7) | 7.8 (0.8) |
| (mg/dl) | | | | |
| Fasting Blood Glucose (mg/dl) | 101.6 (11.9) | 101.2 (15.9) | 102.4 (9.8) | 93.3 (12.4) |

Experimental Protocol

The study was a randomized, double blind placebo-controlled design consisting of an eight week treatment period. Participants in Groups 1 and 2 were randomly allocated either the placebo or the active capsules containing the formula composition shown in Table 1. Participants received two doses daily of the formula composition or placebo for a total of eight weeks.

The capsules were identical in shape color and appearance, with neither the participants not the researcher administering them knowing which capsule each participant received. Participants were asked about side effects during each return visit.

Body weight and body fat percentage were determined in twelve hour fasted participants using a Tanita™ scale. Height was measured with a stadiometer to the nearest 0.5 cm.

Blood samples were collected after a twelve hour overnight fast, into heparinized tubes. The concentrations of total cholesterol, triacylglycerol, HDL-cholesterol, LDL-cholesterol and glucose were measured using commercial diagnostic kits (cholesterol Infinity, triglyceride Infinity, EZ HDL™ cholesterol, EZ LDL™ cholesterol, Glucose Trinder) from SIGMA Diagnostics. Serotonin was measured using an enzyme immunoassay method (Serotonin EIA kit, BioSource Europe S. A., Belgium), creatinine by a modification of the method of Bartels et al., while MDA was measured by a standard established method known in the art.

Statistical Analyses

Paired student's t-test was carried out on the start and end values of the formula composition and placebo groups, as well as between the placebo and formula composition groups. All statistical analyses were carried out using SPSS (Statistical Package for the Social Sciences) software.

Recruitment of Participants

One hundred and twenty participants were recruited for the study by way of radio advertisements and posters. Participants were invited to an information session in which the details of the study were clearly explained. After the information session, they were enrolled in or excluded from the study based on certain criteria. In particular, the inclusion criteria included (1) the participants be healthy males and females, (2) have BMI between 25 and 40, (3) were not following any particular dietary regime prior to the eight week experiment, (4) were willing to follow a prescribed dietary regime, (5) wanted to lose weight, (6) were willing to give blood samples (5 ml) every 2 weeks, and (7) agreed to sign Ethical committee approved consent form. Exclusion criteria excluded anyone who (1) was currently suffering complications of obesity, (2) diabetics, (3) individuals on weight reduction programs, and (4) those individuals with thyroid function problems.

Administration of Formula Composition

Participants of the experiments were told to take one capsule with lots of water two to five minutes before meals. Participants were strongly advised not to take test material on an empty stomach, because it was believed to cause minor discomfort.

Dietary Restriction

Participants in Group 3 were on a supervised balanced diet (2000-2100 Kcal/day) for the duration of the trial.

Blood Collection

Five ml blood samples were collected from each of the participants after an overnight fast at the start and the end of the experimental period. The blood was transferred into vacutainer tubes and serum prepared and stored (200 μl aliquots) at −20° C. until needed for analyses.

Experimental Methods and Blood Analyses

The data for the group of obese individuals on a diet who were given placebos was collected from a separate university controlled study on Irvingia gabonesis and not part of the experiments performed to test the formula composition. However, it is believed that the data are typical of what would be observed of patients on a 2200 kcal a day diet and on a placebo pill.

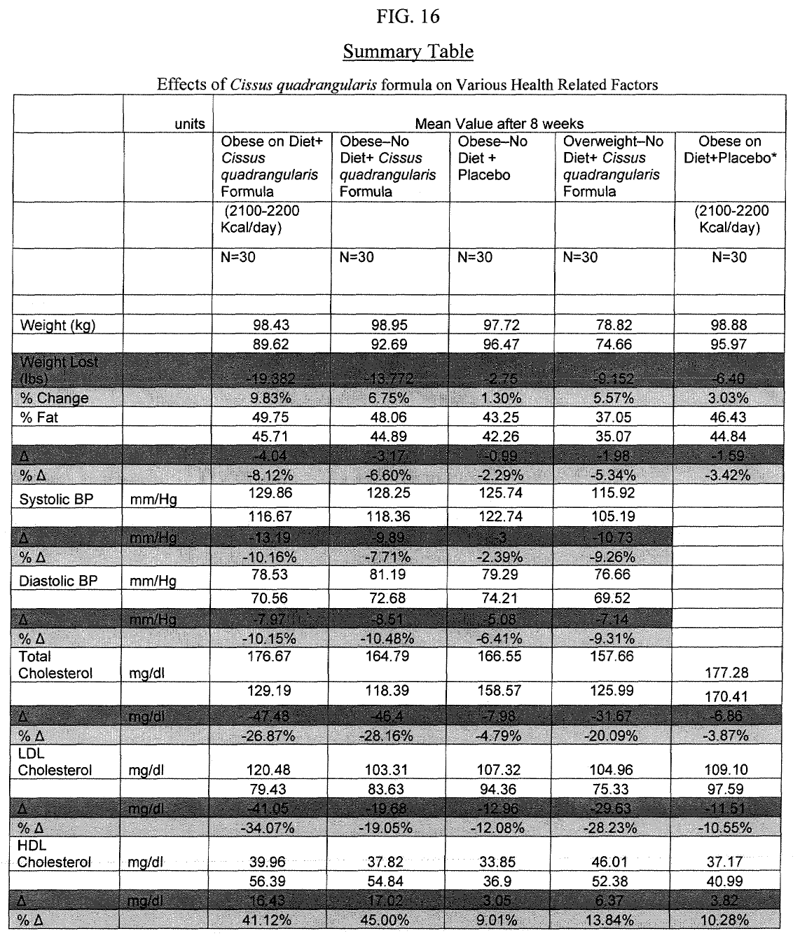

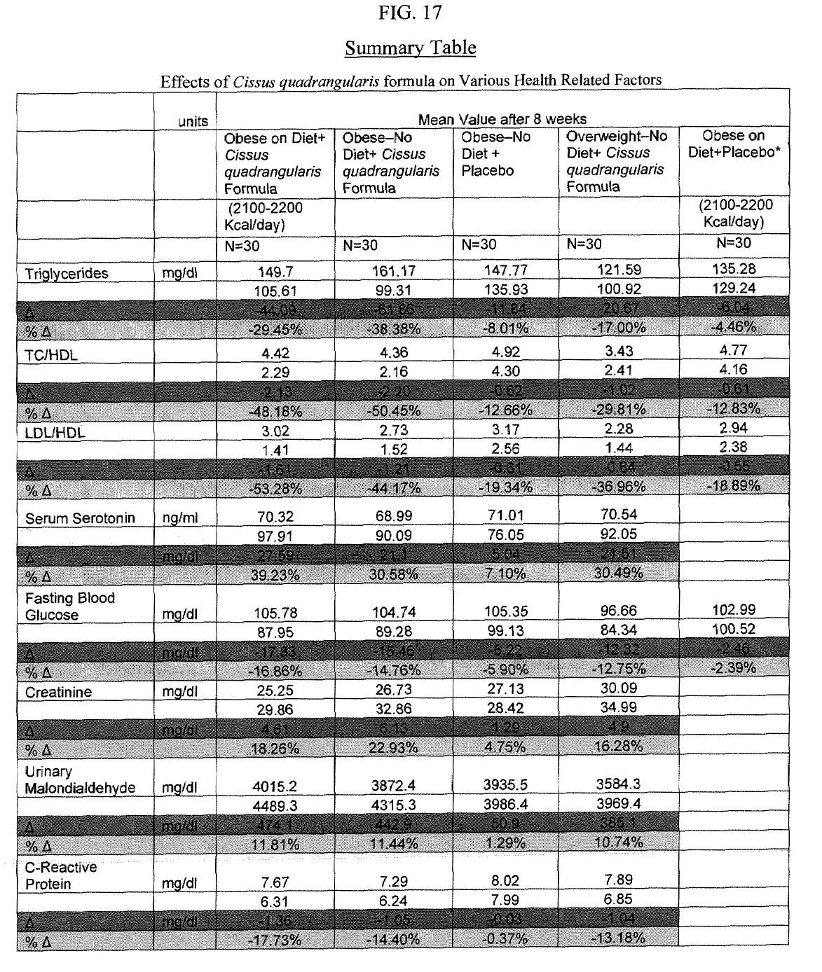

Body fat was estimated by bioelectrical impedance, which measures the body's ability to conduct an electrical current. This conductivity is achieved mainly by lean tissue, implying that body fat measurement actually considers the inverse of lean or muscle tissue mass. Accordingly, the results shown in FIGS. 16 and 17 imply an increase in the ratio of lean to fat tissue in those participants that took the formula composition.

Selected Results

Tables 4 and 5 below show selected results from the experiments showing the effect of the formula composition on a variety of health related factors. The data is shown as a mean with standard deviation in parentheses. For values with an “a” P=<0.001 versus placebo, for values with a “b” P=<0.01 versus placebo, and for values with a “c” P=<0.05 versus placebo.

TABLE 4

| | BMI | | | |
|---|---|---|---|---|
| | | | | 25–29 |
| | Greater than 30 (Obese) | | | (Overweight) |
| | Treatment | | | |
| | | Formulation/ | Formulation/ | Formulation/ |
| Anthropomorphic | Placebo | no diet | Diet | no diet |
| Characteristics* | (n = 30) | (n = 31) | (n = 31) | (n = 31) |
| Weight (%) | −2.4 (4.9) | −6.9 (6.8)^{c} | −8.5 (11.3)^{a} | −4.8 (6.0) |
| Fat (%) | −1.9 (9.6) | −6.0 (8.4) | −8.0 (7.6)^{b} | −4.7 (9.4) |
| Body Mass | −1.0 (4.2) | −8.3 (3.8)^{a} | −9.9 (2.7)^{a} | −3.9 (3.0)^{a} |
| Index (%) | | | | |
| Waist | −2.0 (7.2) | −6.6 (4.9)^{a} | −8.4 (6.7)^{a} | −11.2 (4.7)^{a} |
| Circumference | | | | |
| (%) | | | | |

TABLE 5

| | BMI | | | |
|---|---|---|---|---|
| | | | | 25–29 |
| | Greater than 30 (Obese) | | | (Overweight) |
| | Treatment | | | |
| | | Formulation/ | Formulation/ | Formulation/ |
| Serological | Placebo | no diet | Diet | no diet |
| Characteristics* | (n = 30) | (n = 31) | (n = 31) | (n = 31) |
| Total Cholesterol (%) | −3.1 (28.7) | −27.0 (14.6)a | −26.0 (13.3)a | −18.8 (17.0)b |
| LDL Cholesterol (%) | −10.4 (13.4) | −18.4 (9.8)c | −32.4 (15.5)a | −26.4 (12.8)a |
| HDL Cholesterol (%) | 17.4 (38.8) | 50.5 (50.7)b | 43.0 (39.5)c | 19.6 (37.0) |
| Triglycerides (%) | −4.5 (25.4) | −36.8 (17.8)a | −28.0 (12.2)a | −15.0 (15.3)c |
| C-Reactive Protein (%) | 0.8 (13.2) | −20.8 (11.2)a | −20.8 (10.0)a | −16.3 (13.2)a |
| Fasting Blood Glucose | −4.6 (14.8) | −13.4 (13.6)b | −16.1 (13.4)a | −11.4 (11.8)c |
| (%) | | | | |

FIGS. 1-15 show how the formula composition individually effected the (1) weight, (2) BMI, (3) total cholesterol, (4) LDL cholesterol, (5) HDL cholesterol, (6) total cholesterol/HDL ratio, (7) LDL/HDL ratio, (8) serum serotonin levels, (9) systolic blood pressure, (10) diastolic blood pressure, (11) creatinine levels, (12) urinary malondialdehyde levels, (13) C-reactive protein levels, (14) triglyceride levels, and (15) fasting blood glucose levels for each of the four groups described in Table 2. FIGS. 16 and 17 are tables summarizing all of the results from the experiments on the formula composition's effect on a variety of health related factors, including various cardiovascular and metabolic factors.

It should be appreciated from the data in FIGS. 1, 2, and 16 that the weight lost by obese and overweight participants on the formula composition over the eight week experimental period was, respectively, about seven and three fold more than the weight lost by the placebo group. These results and the others shown and described in FIGS. 1-17 were unexpected and demonstrated the synergistic benefits provided by using the various components of the formula composition together.

Additionally, it should be also be apparent from FIGS. 1-17 that the calorie-restriction in combination with the formula composition synergistically brought a greater weight loss over the eight week period than would have been expected or achieved by combining the effects of the two done separately. It is believed that the higher weight loss by the obese participants compared to overweight participants might have resulted from the obese participants having a higher sensitivity to the formula composition.

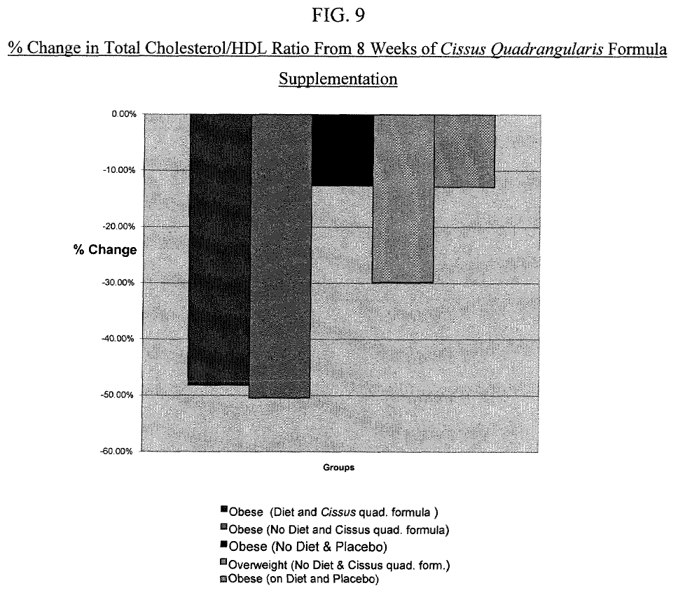

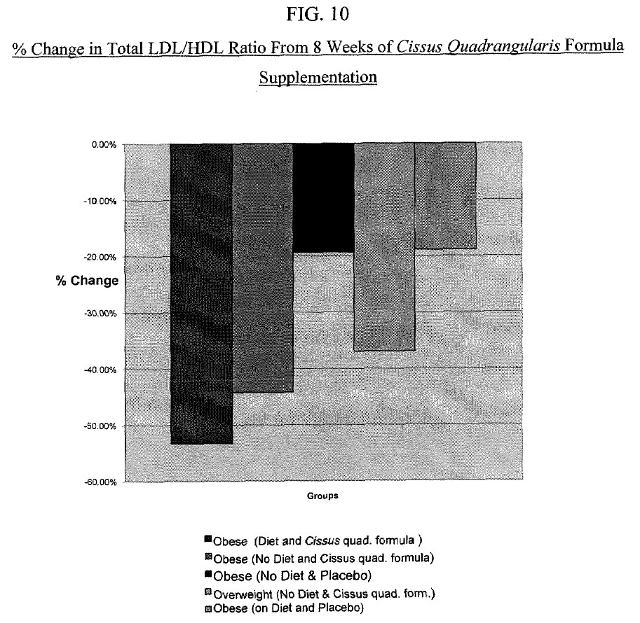

FIGS. 9 and 10 show how the formula composition effected the commonly accepted artherogenicity indices (TC/HDL and LDL/HDL). Both are generally used as indicators of a patient's risk of developing arthrosclerosis. Accordingly, FIGS. 9 and 10 show that the use of the formula composition over an eight week period for the experiments significantly reduced the risk of arthrosclerosis as predicted by a reduction of the artherogenicity index. The reduction of the artherogenicity index was observed as obese as well as overweight participants. It was, however, more pronounced in obese participants as a result of a decrease in either total cholesterol or LDL-cholesterol accompanied by an increase in the concentration of their HDL-cholesterol.

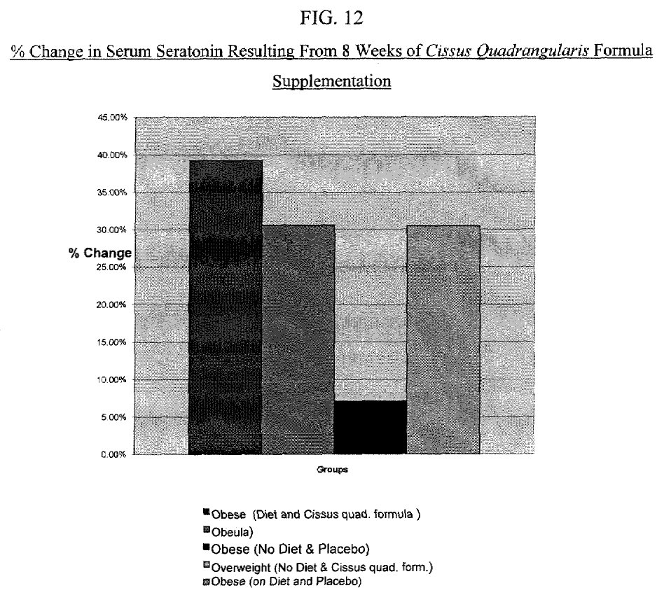

FIG. 12 demonstrates that the formula composition significantly reduced serum serotonin levels in participants. Considering the metabolism of serotonin, these increases are likely to result from a slowing down or inhibition of the re-absorption of circulating serotonin, bringing about satiety which could explain, to an extent, the significant weight loss that accompanies the use of the formula composition.

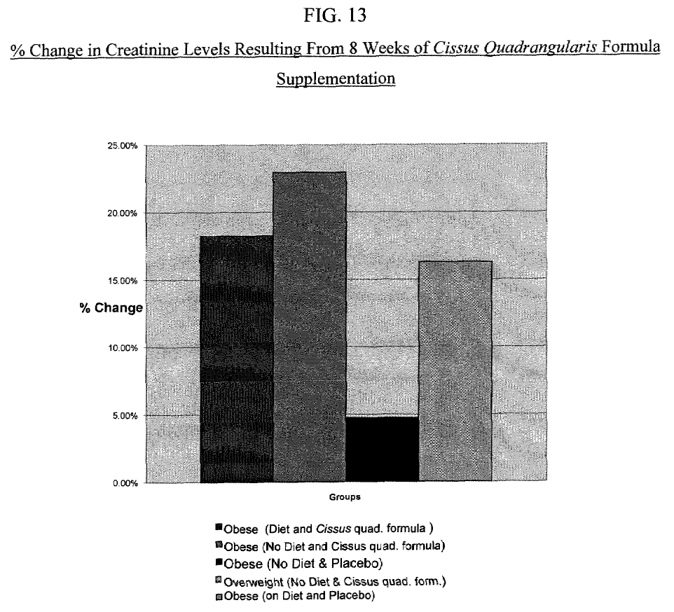

FIG. 13 demonstrates the effect of the formula composition on creatinine levels in the participants. Creatinine is widely used as an indicator of muscle growth. Use of the formula composition for eight weeks significantly increased the circulating concentration of creatinine. This result, corroborates, to an extent, the observation of reduced body fat as well as reduced bio-impedance observed in participants who took the formula composition.

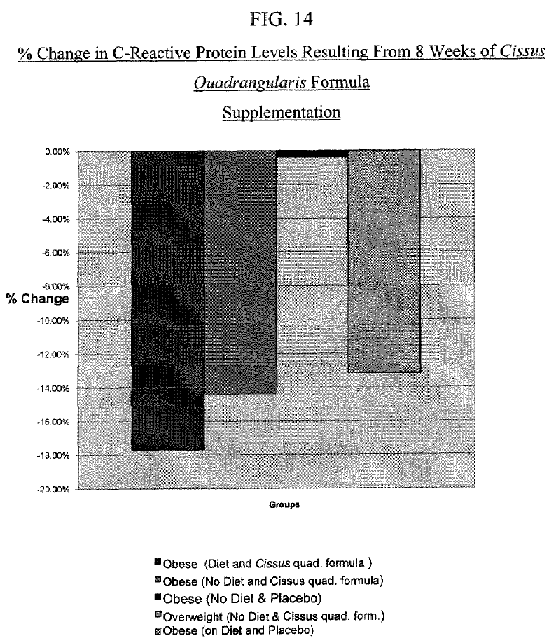

FIG. 14 demonstrates that the formula composition significantly reduces C-reactive protein or CRP. High CRP concentrations are generally considered an indicator of inflammation. Accordingly, the formula composition likely reduces inflammation.

The results from the experiments with 120 human subjects show very clearly that the formula composition, which was well tolerated by users, can be effectively used to improve the numerous health related factors tested and disclosed herein. Importantly, the formula composition significantly reduced body weight as well as body fat. These two factors contribute significantly to the complications associated with being overweight or obese, and are well known risk factors for Syndrome X.

Syndrome X on the other hand, often involves dyslipidemia (hypercholesterolemia, hypertriglyceridemia, increased concentrations of LDL-cholesterol and a decreased concentration of HDL-cholesterol), hyperglycemia and high blood pressure, generally accompanied by oxidative stress and risk of cardiovascular disease. As FIGS. 16 and 17 demonstrate, the formula composition had positive and beneficial effects on the factors that are known to cause Syndrome X, including but not limited to, reducing waist circumference, reducing the fasting blood glucose concentration, reducing hypertriglyceridemia, reducing the circulating concentrations of LDL-cholesterol, increasing the circulating concentrations of HDL-cholesterol and reducing both systolic and diastolic blood pressures. The formula composition also showed a reduced risk of atherosclerosis as determined by a reduction of the artherogenicity index (total cholesterol/HDL-c or LDL-c/HDL-c).

The ability of the formula composition to increase or maintain the concentration of circulating serotonin gives an indication of its potential as a possible appetite suppressant or satiety agent, a factor that could play a very positive role in the control of calorie intake as well as hyperphagia. This control could lead to weight loss and a reversal of the overweight or obese condition.

Accordingly, the formula composition works in a synergistic fashion to reverse the negative consequences of being overweight or obese and improve a variety of health related factors, some of which cause Syndrome X.

It should be appreciated that the compositions of the present disclosure can be used as nutritional ingredients in a wide variety of dietary supplements and nutraceutical food and beverage products, including but not limited to bars, shakes, powders, pre-packaged foods and other health related products.

The foregoing detailed description of the present disclosure is provided for the purposes of illustration, and is not intended to be exhaustive or to limit the disclosure to the particular embodiments disclosed. The embodiments may provide different capabilities and benefits, depending on the configuration used to implement the key features of the disclosure. Accordingly, the scope of the present disclosure is defined only by the following claims.

What is claimed is:

1. A composition for improving one or more cardiovascular or metabolic risk factors in a mammal comprising:
a composition containing an effective amount of extracts from one or more Cissus quadrangularis plants and soy albumin to improve one or more cardiovascular risk factors in a mammal.

2. A composition of claim 1, wherein the effective amount of the Cissus quadrangularis plant extracts is 100 mg to 900 mg and the effective amount of soy albumin is 10 mg to 400 mg.

3. A composition of claim 1, wherein the effective amount of the Cissus quadrangularis plant extracts provides 2.5 mg to 15 mg of active Cissus quadrangularis and the effective amount of soy albumin provides 50 mg to 200 mg of active soy albumin.

4. A composition of claim 1, wherein the composition further includes effective amounts of chromium and selenium.

5. A composition of claim 4, wherein the effective amount of Cissus quadrangularis plant extracts is 100 mg to 900 mg, the effective amount of soy albumin is 10 mg to 400 mg, the effective amount of chromium is 1 mg to 6 mg and the effective amount of selenium is 5 mg to 100 mg.

6. A composition of claim 4, wherein the composition further includes an effective amount of at least three of the following group: green tea extract, vitamin B6, vitamin B12 and folic acid.

7. A composition of claim 6, wherein the effective amount provides 100 mg to 900 mg of Cissus quadrangularis plant extracts, 10 mg to 400 mg soy albumin, 0.15 mg to 0.6 mg chromium, 0.06 mg to 0.24 mg selenium, 100 mg to 1000 mg green tea extract, 10 mg to 400 mg vitamin B6, 0.01 mg to 1.0 mg vitamin B12 and 0.2 mg to 4.0 mg folic acid daily.

8. A composition of claim 6, wherein the effective amount provides 300 mg of Cissus quadrangularis plant extracts, 100 mg soy albumin, 3 mg niacin-bound chromium, 24 mg selenomethionine, 500 mg green tea extract, 100 mg vitamin B6, 0.1 mg vitamin B12 and 0.8 mg folic acid daily.

9. A composition of claim 1, wherein the composition improves two or more of the following cardiovascular or metabolic risk factors in the mammal:
a. weight;
b. BMI;
c. total cholesterol;
d. LDL cholesterol;
e. HDL cholesterol;
f. total cholesterol/HDL ratio;
g. LDL/HDL ratio;
h. serum serotonin levels;
i. systolic and diastolic blood pressure;
j. creatinine levels;
k. urinary malondialdehyde levels;
l. C-reactive protein levels;
m. triglyceride levels; and
n. fasting blood glucose levels.

10. The composition of claim 1, wherein the composition improves one or more of the cardiovascular or metabolic risk factors by:
a. decreasing weight and BMI;
b. lowering total cholesterol levels;
c. lowering systolic blood pressure;
d. lowering diastolic blood pressure;
e. decreasing LDL cholesterol levels;
f. increasing HDL cholesterol levels;
g. decreasing overall blood pressure;
h. lowering triglyceride levels;
i. increasing serum serotonin levels;
j. lowering fasting blood glucose levels;
k. increasing creatinine levels;
l. lowering C-reactive protein levels; and
m. increasing urinary malondialdehyde levels.

11. A composition of claim 6, wherein the composition improves two or more of the following cardiovascular risk factors in the mammal:
a. weight;
b. BMI;
c. total cholesterol;
d. LDL cholesterol;
e. HDL cholesterol;
f. total cholesterol/HDL ratio;
g. LDL/HDL ratio;
h. serum serotonin levels;
i. systolic and diastolic blood pressure;
j. creatinine levels;
k. urinary malondialdehyde levels;
l. C-reactive protein levels;
m. triglyceride levels; and
n. fasting blood glucose levels.

12. The composition of claim 7, wherein the composition improves cardiovascular or metabolic risk factors by:
a. decreasing weight and BMI;
b. lowering total cholesterol levels;
c. lowering systolic blood pressure;
d. lowering diastolic blood pressure;
e. decreasing LDL cholesterol levels;
f. increasing HDL cholesterol levels;
g. decreasing overall blood pressure;
h. lowering triglyceride levels;
i. increasing serum serotonin levels;
j. lowering fasting blood glucose levels;
k. increasing creatinine levels;
l. lowering C-reactive protein levels; and
m. increasing urinary malondialdehyde levels.

13. The composition of claim 11, wherein improving one or more of the cardiovascular risk factors treats Syndrome X or prevents the onset of Syndrome X.

14. The composition of claim 1, wherein improving one or more metabolic risk factors treats Syndrome X or prevents the onset of Syndrome X.

15. The composition of claim 4, wherein the composition further treats Syndrome X or prevents the onset of Syndrome X by eliminating one or more cardiovascular or metabolic risk factors that cause Syndrome X.

16. The composition of claim 6, wherein the composition further treats Syndrome X or prevents the onset of Syndrome X by eliminating one or more cardiovascular or metabolic risk factor that cause Syndrome X.

17. A method for improving or eliminating cardiovascular or metabolic risk factors comprising:
a. providing a composition containing an effective amount of extracts from one or more Cissus quadrangularis plants and soy albumin to a mammal to improve or eliminate one or more cardiovascular or metabolic risk factors; and
b. claiming that the composition improves or eliminates one or more cardiovascular or metabolic risk factors in a mammal.

18. A method of claim 17, wherein the effective amount of Cissus quadrangularis plant extracts in the composition provided to the mammal is 100 mg to 600 mg daily and the effective amount of soy albumin in the composition provided to a mammal is 10 mg to 400 mg daily.

19. A method of claim 17 wherein the effective amount of the Cissus quadrangularis plant extracts in the composition provided to the mammal is 300 mg daily and the effective amount of soy albumin in the composition provided to the mammal is 100 mg daily.

20. A method of claim 19 wherein extracts of the Cissus quadrangularis plants and soy albumin improve or eliminate cardiovascular or metabolic risk factors by reducing weight, total cholesterol, LDL cholesterol, systolic, diastolic and total blood pressure, triglyceride levels, and fasting blood glucose levels and increasing HDL cholesterol, urinary malondialdehyde serum serotonin, and creatinine levels in the mammal.

21. A method of claim 17 wherein the cardiovascular or metabolic risk factors include one or more of the following:
a. weight and BMI;
b. total cholesterol levels;
c. high cholesterol levels;
d. systolic blood pressure;
e. diastolic blood pressure;
f. LDL cholesterol levels;
g. HDL cholesterol levels;
h. overall blood pressure;
i. triglyceride levels;
j. serum serotonin levels;
k. fasting blood glucose levels;
l. creatinine levels;
m. C-reactive protein levels; and
n. urinary malondialdehyde levels.

22. A method for improving or eliminating cardiovascular or metabolic risk factors comprising:
a. providing a composition containing an effective amount of extracts from one or more Cissus quadrangularis plants, soy albumin, chromium and selenium to a mammal to improve or eliminate one or more cardiovascular or metabolic risk factors; and
b. claiming that the composition improves or eliminates one or more cardiovascular or metabolic risk factors in a mammal.

23. A method of claim 22 wherein the effective amount of Cissus quadrangularis plant extracts in the composition provided to the mammal is 100 mg to 600 mg daily, the effective amount of soy albumin in the composition provided to the mammal is 10 mg to 400 mg daily, the effective amount of chromium in the composition provided to the mammal is 0.15 mg to 0.6 mg daily and the effective amount of selenium in the composition provided to the mammal is 0.06 mg to 0.24 mg daily.

24. A method of claim 22 wherein the composition improves or eliminates cardiovascular risk factors by reducing weight, total cholesterol, LDL cholesterol, systolic, diastolic and total blood pressure, triglyceride levels, and fasting blood glucose levels, and increasing HDL cholesterol, urinary malondialdehyde, serum serotonin, and creatinine levels in the mammal.

25. A method of claim 22 wherein the composition improves or eliminates cardiovascular or metabolic risk factors by causing three or more of the following to occur:
a. decreasing weight and BMI;
b. lowering total cholesterol levels;
c. lowering systolic blood pressure;
d. lowering diastolic blood pressure;
e. decreasing LDL cholesterol levels;
f. increasing HDL cholesterol levels;
g. decreasing overall blood pressure;
h. lowering triglyceride levels;
i. increasing serum serotonin levels;
j. lowering fasting blood glucose levels;
k. increasing creatinine levels;
l. lowering C-reactive protein levels; and
m. increasing urinary malondialdehyde levels.

26. A method of claim 17 wherein the cardiovascular or metabolic risk factors include one or more of the following:
a. weight and BMI;
b. total cholesterol levels;
c. high cholesterol levels;
d. systolic blood pressure;
e. diastolic blood pressure;
f. LDL cholesterol levels;
g. HDL cholesterol levels;
h. overall blood pressure;
i. triglyceride levels;
j. serum serotonin levels;
k. fasting blood glucose levels;
l. creatinine levels;
m. C-reactive protein levels; and
n. urinary malondialdehyde levels.

27. A method for improving or eliminating cardiovascular or metabolic risk factors comprising:
a. providing a composition containing an effective amount of extracts from one or more Cissus quadrangularis plants, soy albumin, niacin-bound chromium, selenium, and at least two components selected from the group of green tea extract, vitamin B6, vitamin B12 and folic acid, to a mammal to improve or eliminate one or more cardiovascular risk factors; and
b. claiming that the composition improves or eliminates one or more cardiovascular or metabolic risk factors in a mammal.

28. A method of claim 27 wherein the effective amount of Cissus quadrangularis plant extracts in the composition provided to the mammal is 100 mg to 600 mg daily, the effective amount of soy albumin in the composition provided to the mammal is 10 mg to 400 mg daily, the effective amount of chromium provided to the mammal is 0.15 mg to 0.6 mg daily, the effective amount of selenium provided to the mammal is 0.06 mg to 0.24 mg daily, the effective amount of green tea extract provided to the mammal is 100 mg to 1000 mg daily, the effective amount of vitamin B6 provided to the mammal is 10 mg to 400 mg daily, the effective amount of vitamin B12 provided to the mammal is 0.01 mg to 1.0 mg daily and the effective amount of folic acid provided to the mammal is 0.2 mg to 4.0 mg daily.

29. A method of claim 28 wherein the composition improves or eliminates cardiovascular risk factors by reducing weight, total cholesterol, LDL cholesterol, systolic, diastolic and total blood pressure, triglyceride levels and fasting blood glucose levels and increasing HDL cholesterol, urinary malondialdehyde, serum serotonin, and creatinine levels in the mammal.

30. A method of claim 27 wherein the composition improves or eliminates cardiovascular or metabolic risk factors by causing three or more of the following to occur:
a. decreasing weight and BMI;
b. lowering total cholesterol levels;
c. lowering systolic blood pressure;
d. lowering diastolic blood pressure;
e. decreasing LDL cholesterol levels;
f. increasing HDL cholesterol levels;
g. decreasing overall blood pressure;
h. lowering triglyceride levels;
i. increasing serum serotonin levels;
j. lowering fasting blood glucose levels;
k. increasing creatinine levels;
l. lowering C-reactive protein levels;
m. increasing urinary malondialdehyde levels.

31. A method of claim 29 wherein the cardiovascular or metabolic risk factors include one ore more of the following:
a. weight and BMI;
b. total cholesterol levels;
c. high cholesterol levels;
d. systolic blood pressure;
e. diastolic blood pressure;
f. LDL cholesterol levels;
g. HDL cholesterol levels;
h. overall blood pressure;
i. triglyceride levels;
j. serum serotonin levels;
k. fasting blood glucose levels;
l. creatinine levels;
m. C-reactive protein levels; and
n. urinary malondialdehyde levels.

32. A method for improving or eliminating the metabolic risk factors that cause Syndrome X comprising:
a. providing a composition containing an effective amount of extracts of one or more Cissus quadrangularis plants and soy albumin to a mammal to improve or eliminate one or more metabolic risk factors that cause Syndrome X; and
b. claiming that the composition improves or eliminates Syndrome X or one or more of the metabolic risk factors that cause Syndrome X in a mammal.

33. A method of claim 32 wherein the effective amount of Cissus quadrangularis plant extracts in the composition provided to the mammal is 100 mg to 600 mg daily and the effective amount of soy albumin in the composition provided to the mammal is 10 mg to 400 mg daily.

34. A method of claim 33 wherein the composition further includes green tea extract, vitamin B6, vitamin B12 and folic acid.

35. A method of claim 34 wherein the composition improves or eliminates one or more metabolic risk factors that cause Syndrome X by:
a. decreasing weight and BMI;
b. lowering total cholesterol levels;
c. lowering systolic blood pressure;
d. lowering diastolic blood pressure;
e. decreasing LDL cholesterol levels;
f. increasing HDL cholesterol levels;
g. decreasing overall blood pressure;
h. lowering triglyceride levels;
i. increasing serum serotonin levels;
j. lowering fasting blood glucose levels;
k. increasing creatinine levels;
l. lowering C-reactive protein levels; and
m. increasing urinary malondialdehyde levels.

36. A method of claim 34 wherein the metabolic risk factors include one or more of the following:
a. weight and BMI;
b. total cholesterol levels;
c. high cholesterol levels;
d. systolic blood pressure;
e. diastolic blood pressure;
f. LDL cholesterol levels;
g. HDL cholesterol levels;
h. overall blood pressure;
i. triglyceride levels;
j. serum serotonin levels;
k. fasting blood glucose levels;
l. creatinine levels;
m. C-reactive protein levels; and
n. urinary malondialdehyde levels.

37. A composition reducing weight in a mammal comprising:
a composition containing an effective amount of extracts from one or more Cissus quadrangularis plants, soy albumin, chromium, selenium, green tea extract, chromium, and one or more vitamins and to reduce weight in a mammal.

38. A composition of claim 37, wherein the one or more vitamins are vitamin B6 and vitamin B12 and the effective amount provides 100 mg to 900 mg of Cissus quadrangularis plant extracts, 10 mg to 400 mg soy albumin, 0.15 mg to 0.6 mg chromium, 0.06 mg to 0.24 mg selenium, 100 mg to 1000 mg green tea extract, 10 mg to 400 mg vitamin B6, 0.01 mg to 1.0 mg vitamin B12 and 0.2 mg to 4.0 mg folic acid daily.

39. A method for reducing weight in a mammal comprising:
a. providing a composition containing an effective amount of extracts from one or more Cissus quadrangularis plants, soy albumin, chromium, selenium, green tea extract, chromium, and one or more vitamins to a mammal to improve or eliminate one or more cardiovascular or metabolic risk factors; and
b. claiming that the composition improves or eliminates one or more cardiovascular or metabolic risk factors in a mammal.

40. A method of claim 22 wherein the one or more vitamins are vitamin B6 and vitamin B12 and the effective amount of Cissus quadrangularis plant extracts in the composition provided to the mammal is 100 mg to 600 mg daily, the effective amount of soy albumin in the composition provided to the mammal is 10 mg to 400 mg daily, the effective amount of chromium in the composition provided to the mammal is 0.15 mg to 0.6 mg daily and the effective amount of selenium in the composition provided to the mammal is 0.06 mg to 0.24 mg daily.
